(19) United States
(12) Patent Application Publication
(10) Pub. No.: US 2025/0349404 A1
(43) Pub. Date: Nov. 13, 2025

(54) METHODS AND SYSTEMS FOR CREATING MEDICAL REPORT TEXTS

(71) Applicant: Smart Reporting GmbH, München (DE)

(72) Inventors: Su Hwan KIM, Munich (DE); Francisco PINTO, Munich (DE); Philipp MATTHIES, München (DE)

(21) Appl. No.: 18/871,647

(22) PCT Filed: Jun. 8, 2022
(86) PCT No.: PCT/EP2022/065567

§ 371 Date: Dec. 4, 2024

(51) Int. Cl. G16H 15/00

(52) U.S. Cl. CPC G16H 15/00

(57) ABSTRACT

Creating a medical report, indicative of a longitudinal examination includes receiving an input longitudinal report data structure having longitudinal data elements, which are associated with annotation elements of a medical annotation database that is indicative of relationships between medical concepts. A set of reference report data structures is received, which has reference data elements associated with annotation elements of the medical annotation database. For each longitudinal data elements, it is determined whether the longitudinal data element can be linked with a reference data element of at least one reference report data structure from the set of reference report data structures. The determining is made based on the associated annotation elements and based on the medical annotation database. An interface element indicative of a pointer from the input longitudinal report data structure to the reference report data structure having the linked reference data element, is outputted responsive to the determining.

### FIELD OF THE INVENTION

This invention relates to the field of creating medical report texts.

### BACKGROUND OF THE INVENTION

Medical reports are drafted by physicians for a variety of medical examinations and procedures. Accurate and detailed documentation is important, for instance for medicolegal reasons as well as for sharing and distributing relevant clinical information to medical staff involved in the care of the respective patient.

When drafting a medical report, e.g., of a patient's diagnostic examination, physicians summarize their observations made and the conclusions drawn as well as actions to be followed (e.g., further diagnostics, recommendations). Patients may undergo another examination for evaluating the temporal development of the same condition (a so-called follow-up examination) or they will undergo subsequent examinations for other medical conditions. As a result, multiple medical reports for a given patient may be created over the course of time. Any subsequent report (whether intended for the same condition or for another condition) may be referred to as a longitudinal report. Examples of longitudinal report include follow-up reports.

Upon such subsequent (i.e. longitudinal) examination, a physician (who may be an individual that has performed some of the previous examinations or who may be another individual that has not performed any of the previous examinations) will again draft a medical report on the subsequent examination, i.e., a medical longitudinal report. As the medical history typically contains valuable information which may inform the conclusions or recommendations of the longitudinal examination, the creation of any subsequent report of the patient may be informed by the content of any previous report of the patient.

Various approaches have been suggested to create a longitudinal report in general and follow-up reports in particular. Conventionally, the longitudinal report may have been created completely without formalized reference to any previous report. Such approaches have led to the creation of isolated documents for each examination, with diverging structures and thus poor analyzability over the course of the medical history of the patient.

Another approach for creating a follow-up report may have been for the follow-up physician to briefly scan over a multitude of previous reports and manually select one or more of them for reference. Yet, due to lack of time, it is often not possible or appropriate for users to go through every single report that may potentially contain relevant information.

Such approaches are thus prone to errors in that the physician might overlook a relevant previous report among the multitude of previous reports and valuable information over the course of time may get lost or overlooked. Against this background, there is a need to improve the creation of medical reports indicative of a longitudinal examination of a patient.

### SUMMARY OF THE INVENTION

The invention provides methods, systems, and computer program products to improve the creation of medical report texts indicative of a longitudinal examination of a patient. Aspects of the invention are defined in the independent claims. The dependent claims relate to preferred embodiments.

One aspect of the invention concerns a method for creating a medical report which is indicative of a longitudinal examination of a patient.

The method comprises receiving an input longitudinal report data structure having longitudinal data elements. The longitudinal data elements (which may also be referred to as longitudinal report data elements) are associated with annotation elements of a medical annotation database, wherein the database is indicative of relationships between medical concepts.

The method further comprises receiving a set of one or more reference report data structures of the patient, wherein each reference report data structure has reference data elements associated with annotation elements of said medical annotation database.

The method further comprises determining, for each of the longitudinal data elements, whether said longitudinal data element can be linked with a reference data element of at least one reference report data structure from the set of reference report data structures.

The determining is made based on the associated annotation elements and based on the medical annotation database.

The method also comprises outputting, responsive to the determining, an interface element indicative of a pointer from the input longitudinal report data structure to said reference report data structure having said linked reference data element.

In some embodiments, the longitudinal data elements are indicative of at least one or more of the following: a clinical question of the longitudinal examination; a medical finding of the longitudinal examination; a report date of the longitudinal examination; and a diagnostic procedure of the longitudinal examination.

In some embodiments, the interface element is configured to at least partially display the reference data elements or the linked reference report data structure. For instance, the interface element may be configured to indicate one or more of the following:
a value or content of the reference data element;
a title of said linked reference report data structure;
a date of said linked reference report data structure;
an author indicated by said linked reference report data structure;
one or more multimedia files, such as images, associated with said linked reference report data structure;
a diagnostic procedure related to said linked reference report data structure; and
a therapeutic procedure related to said linked reference report data structure.

In some embodiments, the method further comprises generating a graphical or textual indication. The indication is indicative of a temporal evolution of values or contents of the data elements, which evolution comprises the longitudinal data element and said linked reference data element.

In some embodiments, the method further comprises receiving user input in response to the output interface element, and including the pointer in the longitudinal report data structure in response to the received user input.

In some embodiments, the pointer from the input longitudinal report data structure to said reference report data structure may be a pointer from the longitudinal data element to the linked reference data element.

In some examples, the method further comprises:
determining whether the data structures comprise further pointers, from the input longitudinal data element or from said linked reference data element, to further linked data elements; and
creating, responsive to said determining of further pointers, a set of said data elements which are linked by at least one of said pointer or said further pointers.

In some embodiments, the interface element is output in the longitudinal report data structure. For instance, it may be output as a hyperlink indicative of the pointer.

In some embodiments, the interface element is output as a user interface element requiring confirmation by a user.

In some embodiments, the determining is based on a relationship between the associated annotation elements encoded in the medical annotation database.

For instance, the determining may be based on a clinical relationship between the associated annotation elements, wherein the medical annotation database indicates whether the annotation element associated with the longitudinal data element and the annotation element associated with the reference data element are related to a same clinical question.

For instance, the determining may further be based on a causality relationship between the associated annotation elements, wherein the medical annotation database indicates whether the annotation element associated with the longitudinal data element is a potential consequence of the annotation element associated with the reference data element.
For instance, the determining may further be based on a statistical relationship between the associated annotation elements, wherein the medical annotation database indicates whether the annotation element associated with the longitudinal data element is statistically correlated with the annotation element associated with the reference data element.
For instance, the determining may further be based on the presence of a regional relationship between the associated annotation elements, wherein the annotation element associated with the longitudinal data element and the annotation element associated with the reference data element are related if they refer to a same anatomical region.

For instance, the determining may further be based on the presence of a modality relationship between the associated annotation elements, wherein the annotation element associated with the longitudinal data element and the annotation element associated with the reference data element are related if they refer to a same examination modality

In some embodiments, the determining is further based on the presence of a temporal relationship between the data elements, wherein the data elements are related if a time between the date of the reference examination indicated by the reference report data structure and the date of the longitudinal examination does not exceed a threshold duration, predetermined for the annotation element associated with the longitudinal data element and the annotation element associated with the reference data element.

In some examples, the medical annotation database comprises a medical ontology. Additionally, or alternatively the medical annotation database may comprise a graph data structure, such as a graph database or a relational database which has been configured to represent graph-type relations.

In some embodiments, said set of one or more reference report data structures is received in response to a user-definable query, the query comprising requirements for said reference report data structures.

In a second aspect, a computer program product for creating a medical report is provided, the medical report being indicative of a longitudinal examination of a patient, which is stored on a non-volatile storage medium and contains computer-readable instructions for carrying out the steps of the method according to the first aspect.

In a third aspect, a computer system for creating a medical report is provided, the medical report being indicative of a longitudinal examination of a patient. The system comprises a processing unit, an input receiving unit to receive input from a user and to communicate said input to the processing unit, and a displaying unit configured to display information to the user. The processing unit is configured to carry out the steps of the method according to the first aspect.

### BRIEF DESCRIPTION OF THE DRAWINGS

FIG. 1 shows a schematic illustration of the steps of a method according to the invention;

FIG. 2 shows a schematic illustration of a longitudinal report data structure as used in embodiments of the invention;

FIG. 3 shows a schematic illustration of a medical annotation database as used in embodiments of the invention;

FIG. 4 shows a schematic illustration of a reference report data structure as used in embodiments of the invention;

FIG. 5 shows a schematic illustration of data structures and data bases as used in embodiments of the invention;

FIG. 6 shows a schematic illustration of a computer system according to the invention;

FIG. 7 shows a schematic illustration of a user interface with an example of a longitudinal report text before establishing a link to a reference report text;

FIG. 8 shows a schematic illustration of a user interface with interface elements according to embodiments;

FIG. 9 shows a schematic illustration of a user interface with interface elements according to embodiments;

FIG. 10 shows a schematic illustration of a user interface with an example of a longitudinal report text after establishing a link to a reference report text;

FIG. 11 shows a schematic illustration of another example of an annotation database and reports associated therewith;

FIG. 12 shows a schematic illustration of another example of an annotation database and reports associated therewith; and

FIG. 13 shows a schematic illustration of another example of an annotation database and reports associated therewith.

### DETAILED DESCRIPTION

The order of any of the steps described in the following can be changed where appropriate. Furthermore, the steps can be performed individually or in combination with each other, where appropriate. All the method steps described can be performed or implemented on a computer system.

One aspect of the invention concerns a computer-implemented method for creating a medical report, the medical report that is indicative of a longitudinal examination of a patient.

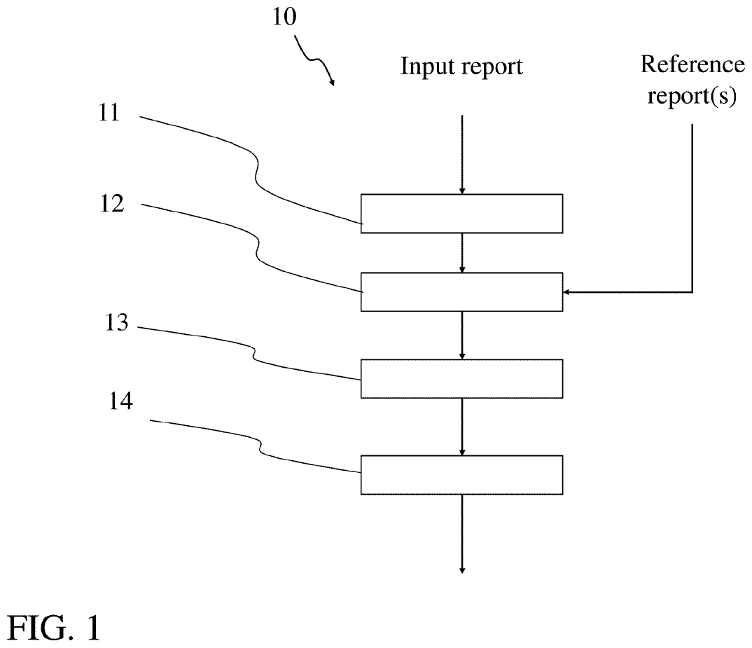

FIG. 1 shows a schematic illustration of the steps of a method 10, which may be computer-implemented. The method 10 is for creating a medical report which is indicative of a longitudinal examination of a patient.

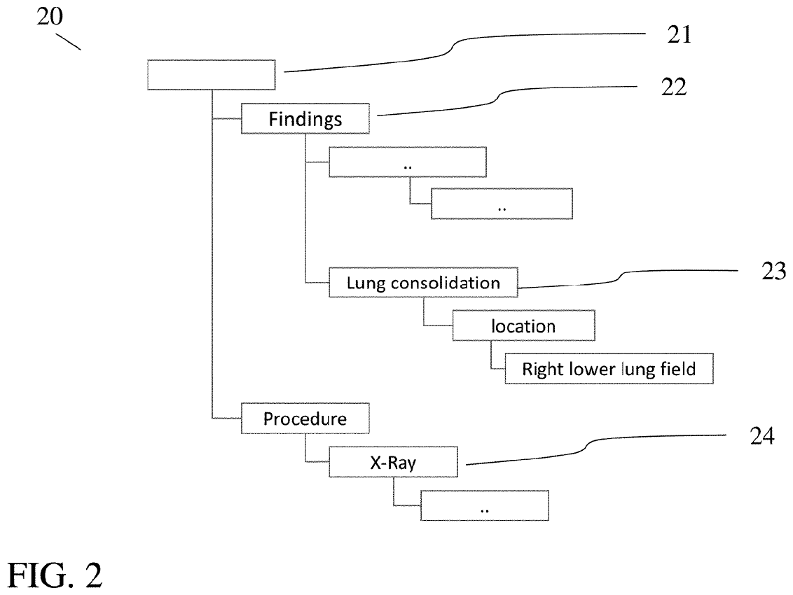

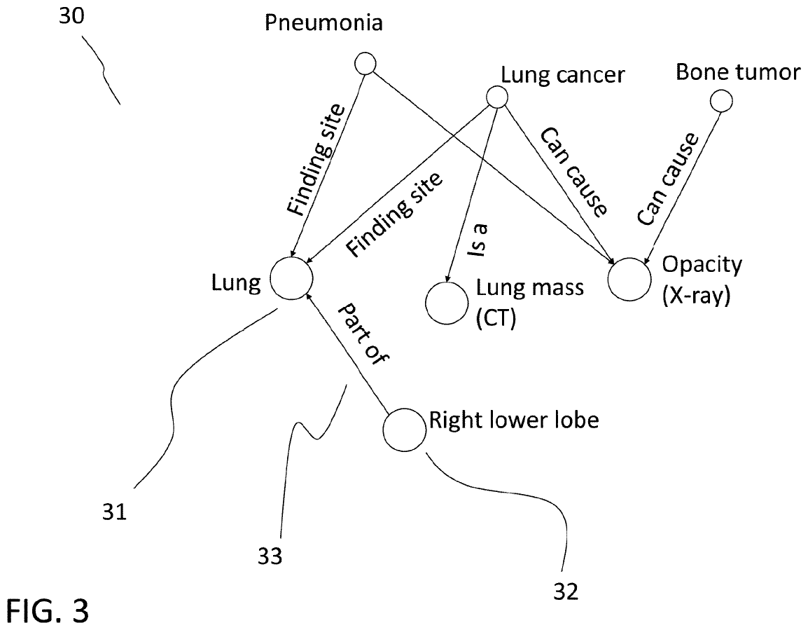

The method 10 comprises a step 11 of receiving an input longitudinal report data structure having longitudinal data elements. An example of a longitudinal report data structure will be described further below with reference to FIG. 2. For instance, examples of longitudinal data elements may be indicative of a clinical question of the longitudinal examination, such as “Exclusion of lung cancer”. Other examples of longitudinal data elements may be indicative of: a medical finding of the longitudinal examination; a report date of the longitudinal examination; or a diagnostic procedure of the longitudinal examination. The longitudinal data elements are associated with annotation elements of a medical annotation database, wherein the database is indicative of relationships between medical concepts. For instance, the medical annotation database may be or may comprise a medical ontology or a graph data structure. An example of a medical annotation database will be described further below with reference to FIG. 3.

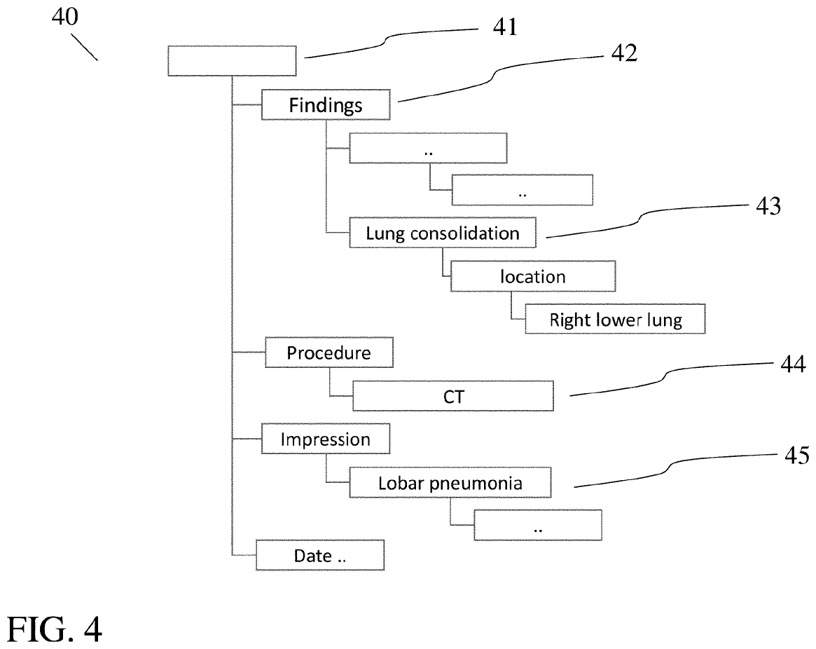

Returning to FIG. 1, the method 10 further comprises a step 12 of receiving a set of one or more reference report data structures of the patient. Each reference report data structure has reference data elements associated with annotation elements of said medical annotation database. An example of a reference report data structure will be described further below with reference to FIG. 4.

In the example of FIG. 1, the receiving relates to any reference report data structure from said patient which is available at a storage device, without further filtering or selection. In other examples, the reference report data structures may first be filtered, e.g., in response to a user-definable query. Such query typically comprises one or more requirements for said reference report data structures, e.g., a date range within which the report must have been created. This allows the user to selectively narrow down the set of reference reports to be used as a basis for the creation of the longitudinal report.

The method 10 further comprises a step 13 of determining, for each of the longitudinal data elements, whether said longitudinal data element can be linked with a reference data element of at least one reference report data structure from the set of reference report data structures.

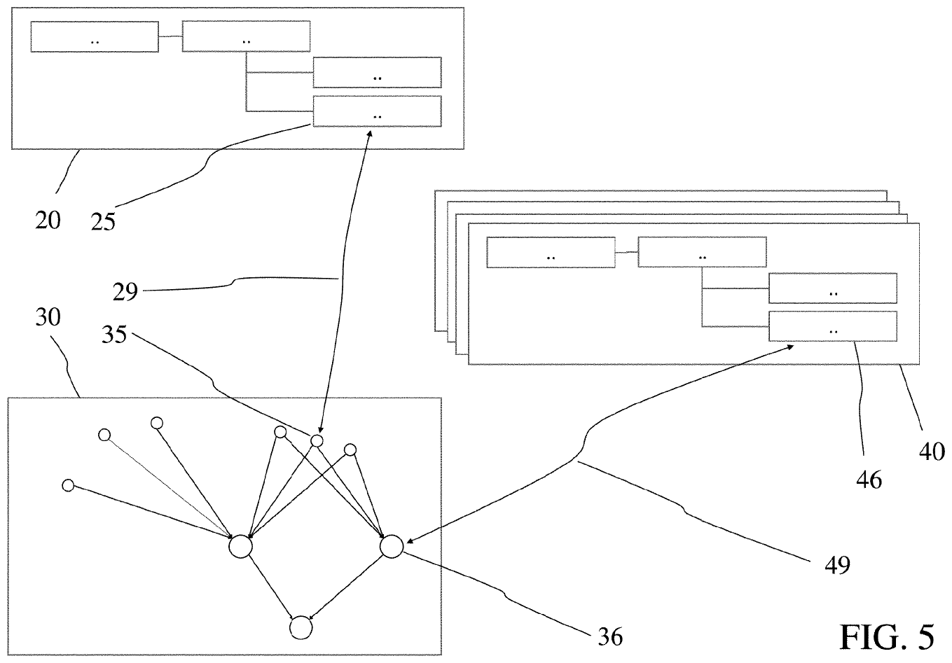

The determining is made based on the associated annotation elements and based on the medical annotation database. Generally, the determining may take into account relationships between the associated annotation elements, which are established by virtue of the medical annotation database, as will be described in further detail below with reference to FIG. 5. As will become apparent from that description, non-limiting examples of such relationships include clinical relationships indicated by the medical annotation database. Other examples include causal or statistical relationships as well as anatomical or modal relationships.

Returning to the description of FIG. 1, the step 13 of determining whether the data elements can be linked allows to generate a pointer from the input longitudinal report data structure to said reference report data structure having said linked reference data element. More specifically, the pointer may be a pointer from the longitudinal data element to the linked reference data element, i.e., may be included on the level of particular data elements rather than more generally on the level of the data structures as such.

The method 10 also comprises a step 14 of outputting, responsive to the determining, an interface element indicative of the generated pointer. For instance, the interface element may be output in the longitudinal report data structure. Examples of such output include hyperlinks indicative of the pointer.

For instance, the interface element may display the reference data elements or the linked reference report data structure. Examples of interface elements may indicate a value or content of the reference data element. Other examples of interface elements may indicate information such as the title of said linked reference report data structure, the creation date of said linked reference report data structure, the author of said linked reference report data structure, one or more multimedia files (e.g., images) associated with said linked reference report data structure, the diagnostic procedure used for said linked reference report data structure, or the therapeutic procedure used for said linked reference report data structure.

In examples, where the interface element is output as a user interface element requiring confirmation by a user, the method 10 may optionally comprise further steps (not illustrated) of receiving user input in response to the output interface element, and of including the pointer in the longitudinal report data structure in response to the received user input. For instance, the received user input may indicate whether the user approves or disapproves of the determined pointer. In response to a positive user input, the pointer may be included, whereas it may be discarded in response to a negative user input. This allows the user to check and confirm whether the determined pointer is accurate. Additionally, or alternatively to such binary user input, the method may also give the user the opportunity to amend or complement the pointer with additional information.

FIG. 2 shows a schematic illustration of a longitudinal report data structure 20. According to the present disclosure, an input longitudinal report data structure having longitudinal data elements is received. For instance, the longitudinal report data structure may be indicative of a longitudinal report text which includes a clinical question and/or one or more medical findings. In some examples, it might not include text (or only include partial text) indicative of the impression of the user about the longitudinal examination. The present disclosure allows to generate suggestions to the user about text to be included in the longitudinal report text. For instance, it may allow to provide suggestions about the text to be included in an “impression” section of the report text, based on the text inputted in the “findings” section. However, the present disclosure shall not be understood to be construed to such particular differentiation between sections of the report text structure. In other examples, the suggestions about text to be included may pertain to the same section as the inputted text.

An example of a longitudinal report data structure is shown in FIG. 2, which includes test elements of an exam report text, structured to reflect the structure of the report text.

Structured report texts, which have been proposed to address the shortcomings of free-text reports, comprise the actual text along with or embedded in a hierarchical structure, such as a hierarchical graph. Contrary to free-text reports, structured reports may be machine-readable and may have an at least partially standardized structure. As a result, they may contain the same elements, wording, and layout as other comparable structured reports. For the generation of structured report, pre-defined report templates can be used, which provide case-specific structure and include recommended reporting steps. Alternatively, a user may select predefined elements, which are then combined to form a report. Further approaches provide for a parsing of a free-form text in order to recognize a structure and mark it accordingly.

Examples of such structures include a hierarchical graph, such as a tree. The nodes (root node, internal nodes and leaf nodes) of such tree are examples of data elements.

Returning to FIG. 2, the data structure 20 is an example of a hierarchical graph. In the example shown, the graph is implemented in the form of a tree structure. A tree data structure represents a directed rooted tree, i.e. a hierarchical data structure with a root, one or more branch nodes and a plurality of leaves.

In the example of FIG. 2, the tree 40 comprises a root 21 with two children (one being designated by reference numeral 22). Child node 22 is a branch node (or internal node), meaning that it has its own children. Other nodes on the other hand do not have further children; they may be referred to as leaf nodes.

The tree 20 comprises data elements such as data element 21. The data elements are related to each other by virtue of parent-child relationships. For instance, data element 22 is a child to data element 21. In the findings sub-tree which is headed by the data element 22 (“Findings”), the longitudinal report data structure 20 comprises various data elements which have been generated in response to the user input in the findings section of the longitudinal report text. In particular, the user has indicated that the x-ray performed as part of the longitudinal examination shows a lung consolidation (data element 23). The location of the lung consolidation is reported as being the “right lower lung field”. Furthermore, the longitudinal report data structure contains data elements indicating for instance the procedure used as part of the longitudinal report examination, namely by “x-ray” examination (data element 24).

The teaching according to the present disclosure makes use of the annotated structure of the longitudinal report text. In some examples, the longitudinal report text may be entered in a structured and annotated fashion by the user directly, for instance via a graphical user interface. In other examples the longitudinal report text may have been entered as a free-form text by the user and this free-form text may have been parsed for terms which indicate data elements which can be annotated using a medical annotation database and parsed for indications of structural delimitations.

The longitudinal data elements may for instance be indicative of a clinical question of the longitudinal examination, such as “Exclusion of lung cancer” (not shown). Other examples of longitudinal data elements may be indicative of: a medical finding of the longitudinal examination (element 23); a report date of the longitudinal examination (not shown); or a diagnostic procedure of the longitudinal examination (element 24).

The longitudinal data elements of longitudinal report data structure 20 are associated with annotation elements of a medical annotation database which is indicative of relationships between medical concepts, as will be described with reference to FIG. 3. For instance, the medical annotation database may be or may comprise a medical ontology or a graph data structure.

In other words, the associated annotation elements may be indicative of a concept of a medical ontology, such that the report text is annotated with medical concepts. Annotated report texts comprise the actual text along with a pointer to or indication of a medical concept in a medical ontology.

Such medical ontology may be specific to a given medical domain, e.g., pulmonology, or it may be a general ontology covering many domains. For instance, databases representing standardized medical ontologies, such as SNOMED CT or ICD-10, may be used. Further examples include “Logical Observation Identifiers Names and Codes” (LOINC) in the field of laboratory medicine, RxNorm in the field of clinical drugs, RadLex in the field of radiology, or “Infectious Disease Ontology” (IDO) in the field of infectious diseases. Such databases may include a vast number of entries (nodes) and relations, such as hundreds of thousands of entries in the current version of SNOMED CT. Furthermore, the content of the knowledge bases to be used may be dynamically updated to reflect the expansion of medical knowledge.

An annotation of a report text allows to enrich the actual text (which is reflective of a particular patient) with more fundamental relations taken from medical knowledge. Such annotation may be based on medical ontologies, with supplementary information. For instance, a pulmonology ontology database may reflect the medical meaning of and relationship between concepts in the field of pulmonology.

FIG. 3 shows a schematic illustration of a medical annotation database 30. In the example shown, the medical annotation database is a graph data structure indicative of a medical ontology.

The medical annotation database 30 comprises various annotation elements, such as annotation element 31 or annotation element 32, which are nodes or vertices (illustrated as circles) of the graph 30. In the example shown, the database is embodied as a graph data structure, while in other examples it may be implemented in another suitable database format such as a relational database. The annotation elements, such as annotation elements 31 and 32, of the annotation database 30 are related to each other by virtue of the edges of the graph. As shown, the edges can be directed, such as from element 32 to element 31 in the case of directed edge 33., which connects annotation elements 31 and 32. In particular it indicates the relationship to be such that the medical concept indicated by annotation element 32 (“right lower lobe”) is “part of” the medical concept indicated by annotation element 31 (“lung”). In other words, the portion of the graph embodied by elements 31, 32 and 33 is indicative of the medical concept/relationship according to which the right lower lobe is part of the lung.

Other elements of the graph 30 indicate knowledge such as the “lung” being the “finding site” of “pneumonia” as well as of “lung cancer”. Furthermore, it indicates that an “opacity” in an x-ray image can be caused by either “lung cancer” or a “bone tumor”. However, only a lung cancer is a “lung mass” in a computer tomography scan.

The example shown in FIG. 3 is only a small portion of the vast medical knowledge which may be encoded in a medical annotation database. As mentioned above, examples of medical annotation databases may contain multiple thousands of entries (nodes), connected by corresponding numbers of relationships (edges). For instance, such medical annotation databases may be based on known databases such as SNOMED CT. Each entry (node) may be identified by a unique annotation element identifier, such as an ontology code. This allows for a unique and easy association between the annotation elements of the medical annotation database (such as data annotation element 31 “lung” of FIG. 3) with a corresponding longitudinal data element of a follow up report (such as longitudinal data element 21 “lung” of FIG. 2).

Non-limiting examples of the relationships (edges) include clinical relationships, such as the “can cause” relationship between lung cancer and opacity as well between bone tumor and opacity. Other examples include causal or statistical relationships as well as anatomical or modal relationships.

The same medical annotation database 30 may be used for associating medical annotation elements with reference report data elements of the set of reference report data structures, as will be explained with reference to FIG. 4.

FIG. 4 shows a schematic illustration of a reference report data structure 40. According to the present disclosure, a set of one or more reference report data structures of the patient is received, wherein each reference report data structure has reference data elements associated with annotation elements of said medical annotation database. An example of a reference report data structure is shown in FIG. 4.

In the example of FIG. 4, the tree-like data structure is similar to the data structure shown in FIG. 2 as an example of an input longitudinal report data structure. However, reference report data structure 40 not only comprises the findings section 42 and a procedure section (cf. data element 44, which indicates the imaging procedure as being a computer tomography). It also comprises an “impression” section, in which the user has entered his impression that the lung consolidation 43 located at a the right lower lung of the CT examination may be indicative of a “lobar pneumonia”, cf. reference report data element 45.

Similar to the input report data structure of FIG. 2, the reference report data elements 41-45 may be associated with the annotation elements of the annotation database 30 of FIG. 3. This allows to determine, for each of the longitudinal data elements, whether said longitudinal data element can be linked with any of the reference data elements of the reference report data structure(s). The determining is made based on the associated annotation elements and based on the medical annotation database. As mentioned above, the determining takes into account relationships (edges) between the associated annotation elements (nodes). On example of such determining will be illustrated with reference to FIG. 5.

FIG. 5 shows a schematic illustration of a longitudinal report data structure 20 (similar to the one of FIG. 2), a medical annotation database 30 (similar to the one of FIG. 3), and multiple reference report data structures 40 (similar to the one of FIG. 4).

The input longitudinal report data structure 20 has longitudinal data elements, such as data element 25. The longitudinal data elements are associated with annotation elements (nodes) of the medical annotation database 30. For instance, data element 25 is associated with annotation element 35 by virtue of the association 29 (for instance by including a reference to the unique annotation identifier of annotation element 35 in the longitudinal report data structure 20).

Each of the set of reference report data structures 40 of the patient has reference data elements (such as data element 46), which are associated with annotation elements of said medical annotation database 30. For instance, data element 46 is associated with annotation element 36 by virtue of the association 49 (for instance by including a reference to the unique annotation identifier and rotation element 36 in the reference report data structure 40).

The annotation database 30 is indicative of relationships between medical concepts. For instance, annotation elements 35 and 36 are related by a corresponding relationship (edge) as illustrated by the arrow between elements 36 and 35 of the graph/database.

The associations 29 and 49, as well as the medical relationship linking elements 35 and 36 in the database 30 allow to establish a link between the longitudinal data element 25 and the reference data element 46 of the reference report data structure 40 from the set of reference report data structures which includes the reference data element 46.

As can be seen from FIG. 5, this link is established based on the associated annotation elements and based on the medical annotation database. Generally, the determining takes into account relationships between the associated annotation elements, which are established by virtue of the medical annotation database. As mentioned above, non-limiting examples of such relationships include clinical relationships indicated by the medical annotation database. Other examples include causal or statistical relationships as well as anatomical or modal relationships.

In response to determining that such link can be established, the present disclosure provides for generating a pointer from the input longitudinal report data structure 20 to said reference report data structure 40 having said linked reference data element 46. More specifically, the pointer may be a pointer from the longitudinal data element 25 to the linked reference data element 46, i.e., may be included on the level of particular data elements 25, 46 rather than more generally on the level of the data structures 20, 40 as such.

Determining whether the data elements can be linked allows to generate a pointer from the input longitudinal report data structure to said reference report data structure having said linked reference data element. The use of medical annotation database 30 to generate the pointer increases the reliability of linking the longitudinal report with suitable reference report, even in case of large numbers of reference reports or in case of diverging examination modalities or diverging nomenclature.

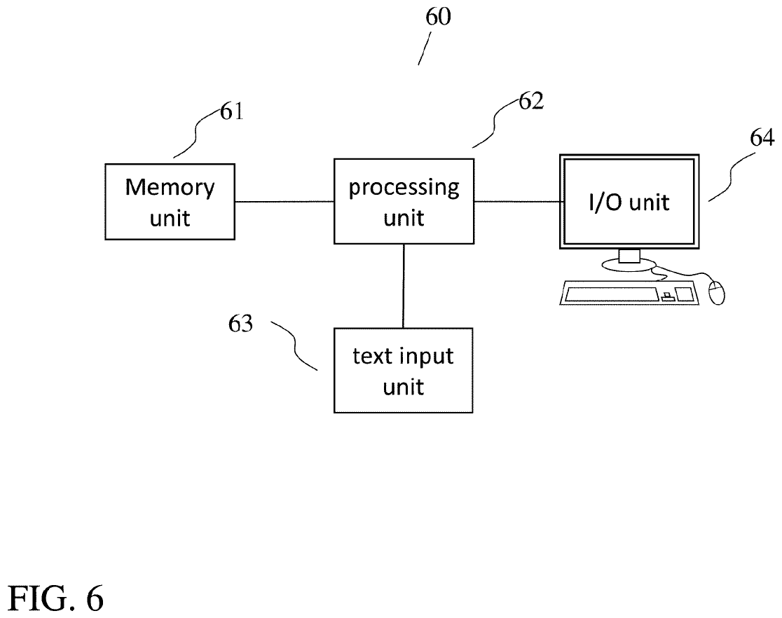

FIG. 6 shows a schematic illustration of a computer system 60. The computer system 60 is for creating a medical report indicative of a longitudinal examination of a patient. The system 60 comprises a memory unit 61, a processing unit 62, an input receiving unit 63, and a displaying unit 64. The memory unit 61 is for storing a set of reference report data structures. The input receiving unit 63 is for receiving input from a user (e.g., input of a longitudinal report text) and to communicate said input to the processing unit 62. The displaying unit 64 is for displaying information to the user. It shall be noted that any of the displayed elements (in particular the memory unit 61 and/or the processing unit 62) may be located locally near the elements of the user terminal or they may be located at another location such as in a remote server. The processing unit 62 is configured to carry out the steps of the method 10 according to FIG. 1.

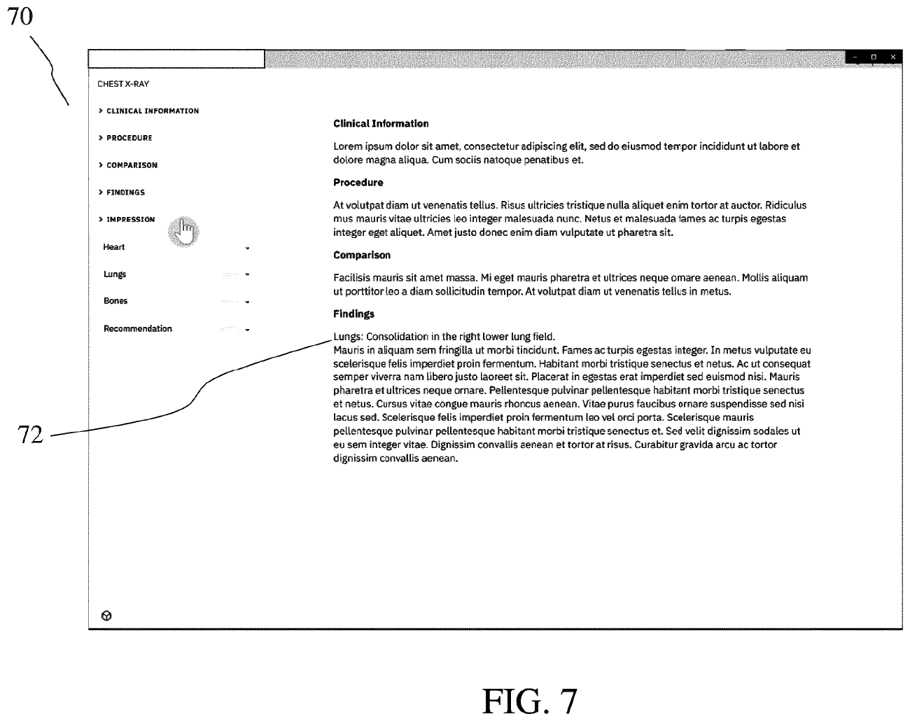

FIG. 7 shows a schematic illustration of a user interface 70, wherein a user has inputted a longitudinal report text. In addition to various free text entries (placeholder text in FIG. 7), the input longitudinal report text in particular includes the finding of a “consolidation in the right lower lung field”, as indicated by text element 72.

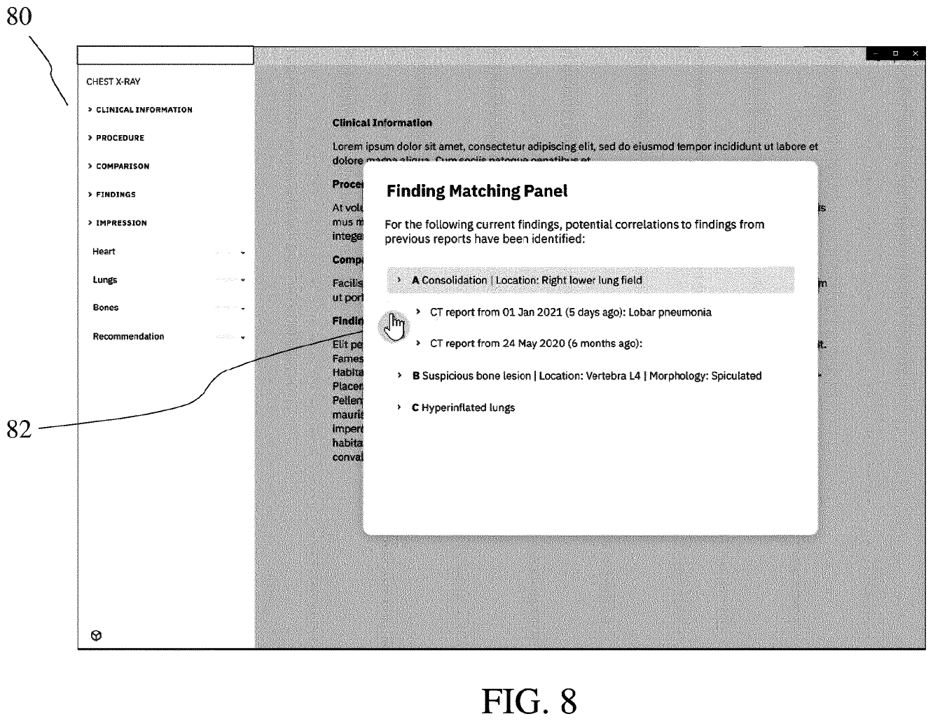

Using the method according to the present disclosure, for instant method 10 of FIG. 1, the present disclosure allows to determine whether said longitudinal data element corresponding to text element 72 can be linked to any of the report reference report data structures on file. In response to this determining, the user interface may output an interface element, as illustrated and described with reference to FIG. 8.

FIG. 8 shows a schematic illustration of a user interface 80. The user interface 80, which may be displayed response in response to determining that one of the longitudinal data elements may be linked to a reference report data element, includes an interface element 82.

In the example shown, the interface element 82 is a pop-up window displaying the reference data elements and the linked reference report data structure. In particular, it indicates the content of the reference data element (“lobar pneumonia”) and the title or modality of said linked reference report data structure (“CT report”), the creation date of said linked reference report data structure (“from 1 Jan. 2021”).

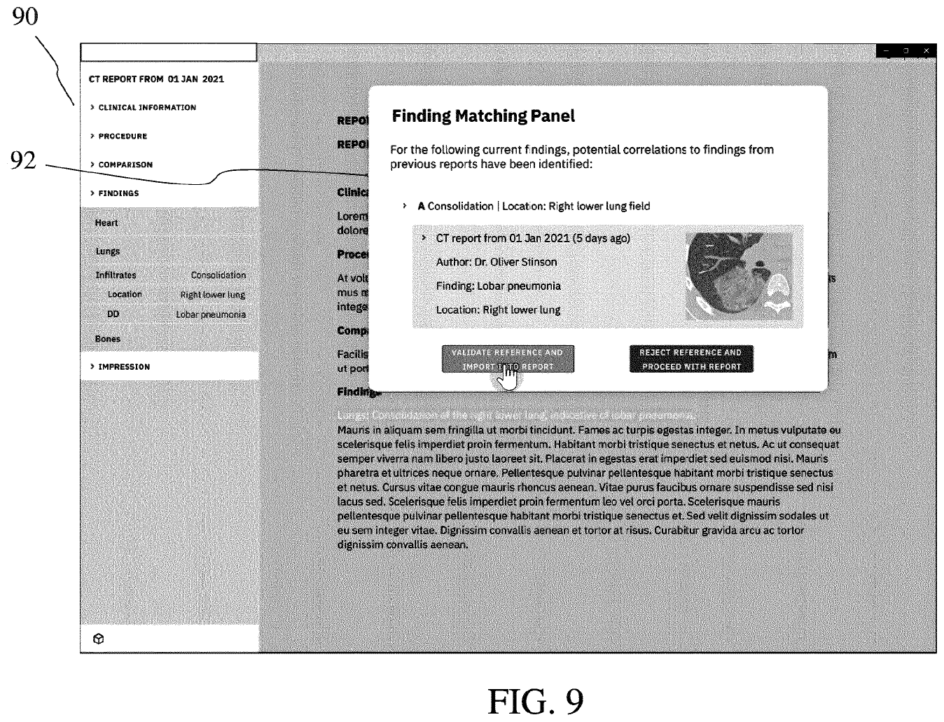

This allows the user to inspect the determined link. For instance the user may select the suggested reference report, as will be described with reference to FIG. 9.

FIG. 9 shows a schematic illustration of a user interface 90, which includes an interface element 92. The interface element 92 is output in order to obtain confirmation by a user that the determined link is to be included into the longitudinal report.

In the example of FIG. 9, the interface element 92 includes further information such as the author of said linked reference report data structure and a snapshot of a multimedia file (here: a snapshot of the CT images) associated with said linked reference report data structure. It also indicates the diagnostic procedure used for said linked reference report data structure (CT). In other embodiments, a graphical or textual indication may be generated, which is indicative of a temporal evolution of values or contents of the data elements, which evolution comprises the longitudinal data element and said linked reference data element.

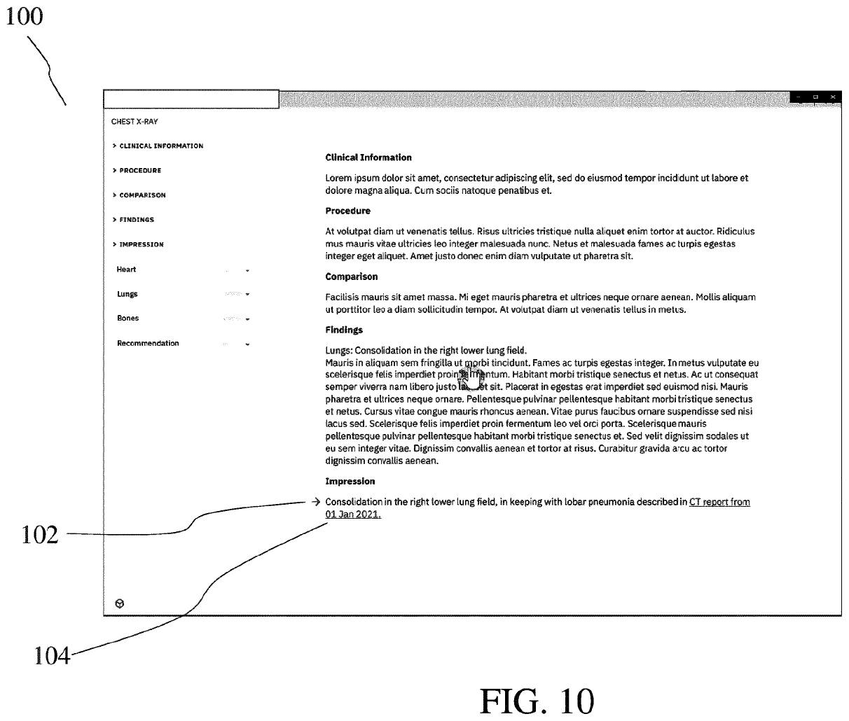

In some embodiments of the present disclosure, user input may be received in response to the output of an interface element such as interface element 90. In response to receiving a confirming user input, the pointer may be included in the longitudinal report data structure. For instance, the received user input may indicate whether the user approves or disapproves of the determined pointer. In response to a positive user input, the pointer may be included, whereas it may be discarded in response to a negative user input. This allows the user to check and confirm whether the determined pointer is accurate. In response to such user input, the user interface may switch back to the input reference report text, now including the pointer as will be described with reference to FIG. 10.

FIG. 10 shows a schematic illustration of a user interface 100, including the input longitudinal report text of FIG. 7. However, in addition to the information provided in FIG. 7, the input longitudinal report text now also includes an indication 102 of the generated pointer in the longitudinal text. In the example shown, this indication includes a hyperlink 104 indicative of the pointer, which allows to directly access the linked reference report text. This indication 100 to is included not only in the report text, as displayed on the user interface, but it is also included in the underlying longitudinal report data structure.

### Example 1

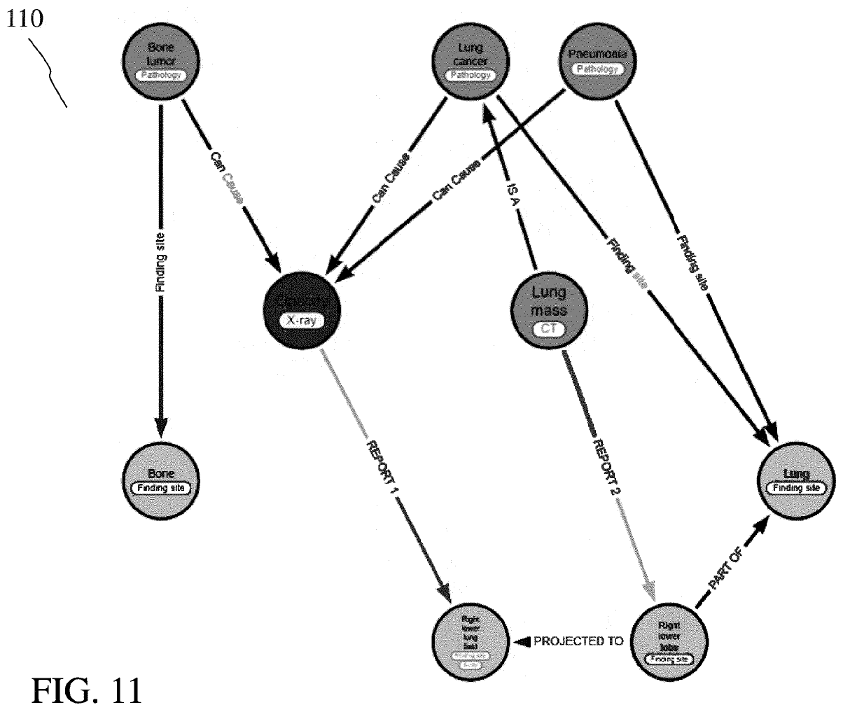

FIG. 11 illustrate a first example of an annotation database and reports associated therewith, for use in the teaching of the present disclosure. For ease of reference, the various data elements or associated annotation elements are indicated by virtue of [square brackets] in the following.

In this first example, the patient has had a first exam, which was a [X-ray] exam of the chest where an unclear [opacity] in the [right lower lung field] was found (cf. “REPORT 1”). As a consequence, a second exam was requested, namely a [CT] examination of the chest for further clarification (cf. “REPORT 2”).

As a first parameter, the clinical question of REPORT 2 allows to establish a link: In such examples, the medical annotation database indicates whether the annotation element associated with the longitudinal data element and the annotation element associated with the reference data element are related to a same clinical question

The clinical question of REPORT 2 was specified as: “Exclusion of [lung cancer]”.

The challenge for the radiologist performing the second exam is to identify the original description of the opacity in REPORT 1 without reviewing all other previous imaging reports. According to the present disclosure, the use of an annotation database allows to identify relevant previous findings from the entered clinical question, which includes the element [Lung cancer].

Based on this information, the method or system according to the example searches through the previous imaging reports of the patients that may potentially be related to the pathology [Lung cancer].

In this specific case, the annotation database contains (inter alia) the following explicit relationships between annotation elements: [Lung cancer] Can Cause [Opacity]; [Lung cancer] Finding Site [Lung]. In other words, the annotation database may indicate whether one of the data elements (e.g., the longitudinal data element) is a potential consequence of another data element (e.g., the reference data element). Similarly, the medical annotation database may indicate whether two annotation elements are statistically correlated. Moreover, the database indicates whether two annotation elements relate to a same anatomical region.

From these relationships modeled in the annotation database, the method or system identifies the reference report data element [opacity] in (reference) REPORT 1 and prompts the user to validate or reject the reference to REPORT 1.

This approach can further be refined by utilizing additional parameters, such as anatomical region, modality, age of the report, etc.:

First, the anatomical region allows to establish a (regional) relationship: In particular, the annotation element associated with the longitudinal data element and the annotation element associated with the reference data element may be considered to be related if they refer to a same anatomical region. In the particular example, if the radiologist describes a [lung mass] in the [right lower lobe] in REPORT 2, the method/system can infer that CT findings in the [right lower lobe] (which is a 3D location) are in X-Ray images “projected to” the [right lower lung field] (which is a 2D location). As a result, previous findings e.g. in the left lung can be excluded from the suggested references, since the [left lower lobe] would not be “projected to” the [right lower lung field].

In addition, the user may specifically filter previous findings by modality. For instance, the radiologist can specifically configure the system to only look for findings from X-ray examinations in which lung tumors are commonly observed for the first time, while excluding other modalities.

Similarly, a relationship may be established based on the presence of a modality relationship between the associated annotation elements, wherein the annotation element associated with the longitudinal data element and the annotation element associated with the reference data element are considered to be related if they refer to a same examination modality.

Furthermore, the user can configure the query to cover only findings reported within a certain time frame, e.g., within the past 6 months, assuming that older findings will not be relevant for the current clinical question.

Conversely, the data elements may be considered to not be related if a time between the date of the reference examination indicated by the reference report data structure and the date of the longitudinal examination exceeds a threshold duration, predetermined for the annotation element associated with the longitudinal data element and the annotation element associated with the reference data element.

### Example 2

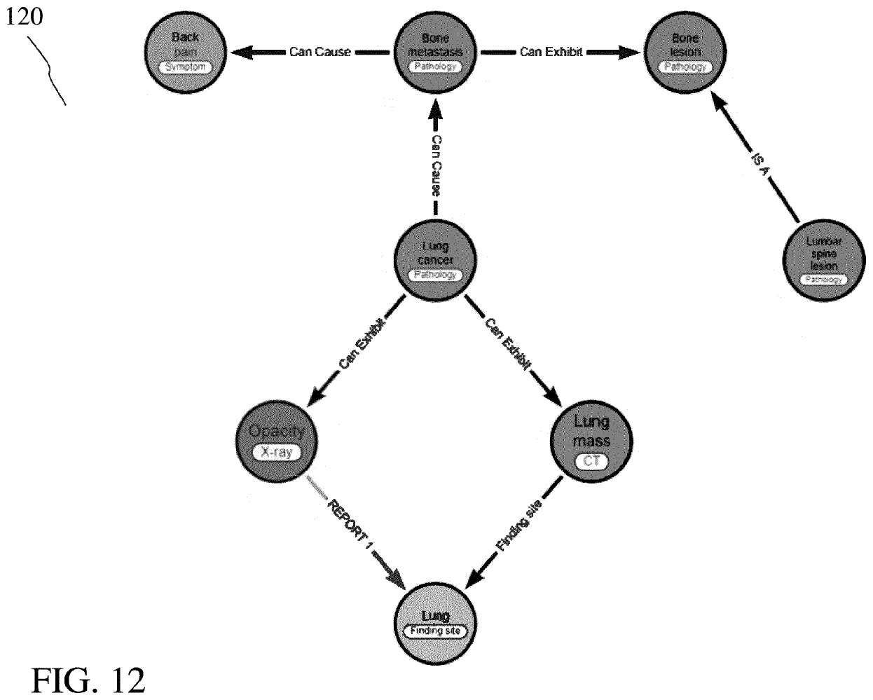

FIG. 12 illustrate a second example of an annotation database and reports associated therewith, for use in the teaching of the present disclosure.

The second clinical scenario deals with the same patient case of the first example, but at a later time point. The patient now presents with acute back pain. To investigate the cause of this symptom, the clinical referrer requests a CT chest/abdomen with the clinical question: “Investigation of acute [back pain]”.

However, the clinical referrer fails to mention to the radiologist that the patient is suffering from lung cancer. Due to the high number of previous reports, there is the risk that the radiologist may overlook that the patient is suffering from lung cancer.

The annotation database contains (inter alia) the following explicit relationships: [Bone metastasis] Can Cause [Back pain]; [Lung cancer] Can Cause [Bone metastasis]; [Lung cancer] Can Exhibit [Opacity]; [Lung cancer] Can Exhibit [Lung mass]; [Lumbar spine lesion] Is A [Bone lesion] (derived from SNOMED CT).

Based on the radiologist's observation of a [lumbar spine lesion] in the current CT examination, the present teaching can suggest the following pathophysiological relationship:

The longitudinal report describes a [bone lesion] in the CT exam. In the reference reports, a [lung mass] in the Chest [CT] exam and an [opacity] in the Chest [X-ray] have been described.

The present teaching can then suggest a potential relationship between the currently observed [bone lesion] and the previous lung findings, which allows the radiologist to realize that the patient is suffering from [lung cancer], without the need to go through all previous exam reports.

This realization has clinical consequences: Bone metastases count as distant metastases, which leads to a change of tumor stage according to the TNM classification for tumor diseases, which indicates a change of treatment plan.

### Example 3

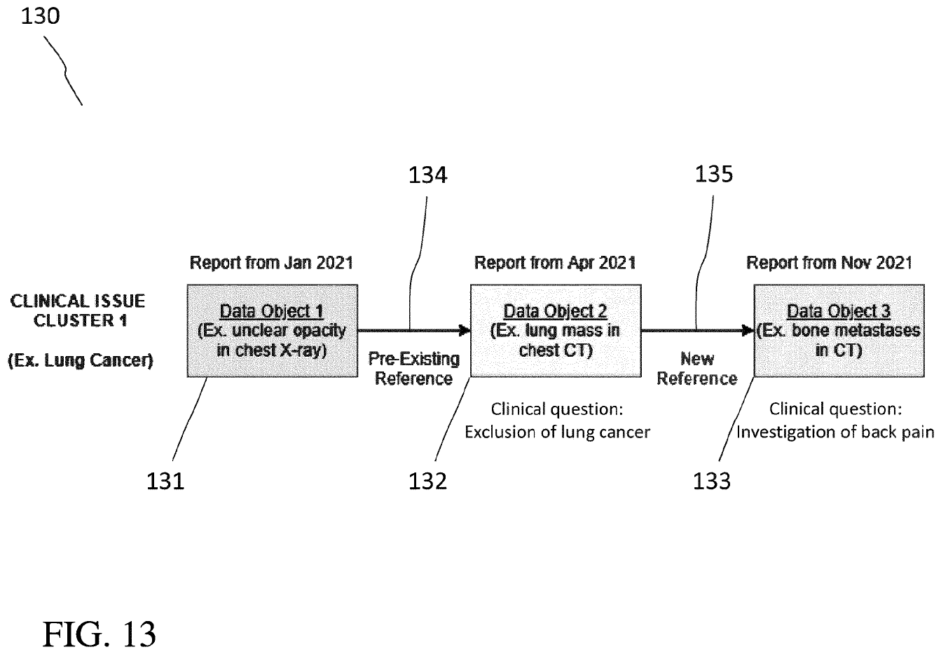

FIG. 13 illustrate a third example of an annotation database and reports associated therewith, for use in the teaching of the present disclosure. The temporal sequence of reports is similar to that of Example 2 above. A first report 131 describes an unclear opacity in an x-ray of the chest. A second report 132 (which may have been conducted as a follow-up examination, specifically following up on the first report 131) describes a lung mass in a computer tomography of the chest, in order to elucidate the clinical question of exclusion of lung cancer. The first report 131 second report will 32 have previously been linked to each other with a reference 134 according to the present disclosure (cf. first example).

Upon creation of a third report 133, which addresses the clinical question of investigation of back pain (and has thus not been conducted specifically as a follow-up to the first and second report 131, 132), this third report 133 may be determined to be a longitudinal report to the second report 132 and a link 135 may be established between the second and third reports 132, 133 (cf. second example).

In addition to establishing this link 135 between the second and third reports, it may be determined whether the data structures comprise further pointers from any of the data elements to further linked data elements. Responsive to the determining of such further pointers, a set of said data elements is created which are linked by at least one of said pointer or said further pointers.

In the example shown, it is determined that one of the data structures, namely the reference report data structure 132, comprises a further pointer, namely reference 134, to further linked data elements in the first report 131. Responsive thereto, a cluster comprising all three reports 131, 132, 133 is created, which reports comprise data elements that are linked by the pointers 134, 135.

1. A method for creating a medical report, the medical report being indicative of a longitudinal examination of a patient, the method comprising
receiving an input longitudinal report data structure having longitudinal data elements, the longitudinal data elements being associated with annotation elements of a medical annotation database, the database being indicative of relationships between medical concepts;
receiving a set of one or more reference report data structures of the patient, wherein each reference report data structure has reference data elements associated with annotation elements of the medical annotation database;
determining, for each of the longitudinal data elements, whether the longitudinal data element can be linked with a reference data element of at least one reference report data structure from the set of reference report data structures, based on the associated annotation elements and based on the medical annotation database; and
outputting, responsive to the determining, an interface element indicative of a pointer from the input longitudinal report data structure to the reference report data structure having the linked reference data element.

2. The method according to claim 1, wherein the longitudinal data elements are indicative of at least one or more of the following:
a clinical question of the longitudinal examination;
a medical finding of the longitudinal examination;
a report date of the longitudinal examination; and
a diagnostic procedure of the longitudinal examination.

3. The method according to claim 1, wherein the interface element is configured to at least partially display the reference data elements or the linked reference report data structure.

4. The method according to claim 3, wherein the interface element is configured to indicate one or more of the following:
a value or content of the reference data element;
a title of the linked reference report data structure;
a date of the linked reference report data structure;
an author indicated by the linked reference report data structure;
one or more multimedia files associated with the linked reference report data structure;
a diagnostic procedure related to the linked reference report data structure; and
a therapeutic procedure related to the linked reference report data structure.

5. The method according to claim 1, further comprising: generating a graphical or textual indication, indicative of a temporal evolution of values or contents of the data elements, the temporal evolution comprising the longitudinal data element and the linked reference data element.

6. The method according to claim 1, further comprising:
receiving user input in response to the output interface element; and
including the pointer in the longitudinal report data structure in response to the received user input.

7. The method according to claim 1, wherein the pointer from the input longitudinal report data structure to the reference report data structure is a pointer from the longitudinal data element to the linked reference data element.

8. The method according to claim 7, further comprising:
determining whether the data structures comprise further pointers, from the input longitudinal data element or from the linked reference data element, to further linked data elements; and
creating, responsive to the determining of further pointers, a set of the data elements which are linked by at least one of the pointer or the further pointers.

9. The method according to claim 1, wherein the interface element is output in the longitudinal report data structure, as a hyperlink indicative of the pointer.

10. The method according to claim 1, wherein the interface element is output as a user interface element requiring confirmation by a user.

11. The method according to claim 1, wherein the determining is based on a clinical relationship between the associated annotation elements, and wherein the medical annotation database indicates whether the annotation element associated with the longitudinal data element and the annotation element associated with the reference data element are related to a same clinical question.

12. The method according to claim 1, wherein the determining is further based on a causality relationship between the associated annotation elements, and wherein the medical annotation database indicates whether the annotation element associated with the longitudinal data element is a potential consequence of the annotation element associated with the reference data element.

13. The method according to claim 1, wherein the determining is further based on a statistical relationship between the associated annotation elements, and wherein the medical annotation database indicates whether the annotation element associated with the longitudinal data element is statistically correlated with the annotation element associated with the reference data element.

14. The method according to claim 1, wherein the determining is further based on one or more of the following:
a presence of a regional relationship between the associated annotation elements, wherein the annotation element associated with the longitudinal data element and the annotation element associated with the reference data element are related if they refer to a same anatomical region;
a presence of a modality relationship between the associated annotation elements, wherein the annotation element associated with the longitudinal data element and the annotation element associated with the reference data element are related if they refer to a same examination modality; and
a presence of a temporal relationship between the data elements, wherein the data elements are related if a time between the date of the reference examination indicated by the reference report data structure and the date of the longitudinal examination does not exceed a threshold duration, predetermined for the annotation element associated with the longitudinal data element and the annotation element associated with the reference data element.

15. The method according to claim 1, wherein the medical annotation database comprises a medical ontology and/or a graph data structure.

16. The method according to claim 1, wherein the set of one or more reference report data structures is received in response to a user-definable query, the query comprising requirements for the reference report data structures.

17. A computer program product for creating a medical report, the medical report being indicative of a longitudinal examination of a patient, which is stored on a non-volatile storage medium and contains computer-readable instructions for carrying out the steps comprising:
receiving an input longitudinal report data structure having longitudinal data elements. the longitudinal data elements being associated with annotation elements of a medical annotation database, the database being indicative of relationships between medical concepts;
receiving a set of one or more reference report data structures of the patient, wherein each reference report data structure has reference data elements associated with annotation elements of the medical annotation database;
determining, for each of the longitudinal data elements, whether the longitudinal data element can be linked with a reference data element of at least one reference report data structure from the set of reference report data structures, based on the associated annotation elements and based on the medical annotation database; and
outputting, responsive to the determining, an interface element indicative of a pointer from the input longitudinal report data structure to the reference report data structure having the linked reference data element.

18. A computer system for creating a medical report, the medical report being indicative of a longitudinal examination of a patient, the system comprising:
a processing unit;
a memory unit to store data;
an input receiving unit to receive input from a user and to communicate the input to the processing unit; and
a displaying unit configured to display information to the user;
wherein the processing unit is configured to:
receive, from the input receiving unit, an input longitudinal report data structure having longitudinal data elements, the longitudinal data elements being associated with annotation elements of a medical annotation database, the database being indicative of relationships between medical concepts;
receive, from the memory unit, a set of one or more reference report data structures of the patient, wherein each reference report data structure has reference data elements associated with annotation elements of the medical annotation database;
determine, for each of the longitudinal data elements, whether the longitudinal data element can be linked with a reference data element of at least one reference report data structure from the set of reference report data structures, based on the associated annotation elements and based on the medical annotation database; and
output, on the displaying unit and responsive to the determining, an interface element indicative of a pointer from the input longitudinal report data structure to the reference report data structure having the linked reference data element.
